(19) United States
(12) Patent Application Publication
(10) Pub. No.: US 2003/0032185 A1
(43) Pub. Date: Feb. 13, 2003

(54) Method of making a homogeneous preparation of hematopoietic stem cells

(76) Inventors: Saul J. Sharkis, Towson, MD (US); Michael I. Collector, Baltimore, MD (US)

Correspondence Address: BANNER & WITCOFF, 1001 G STREET N W, SUITE 1100, WASHINGTON, DC, 20001, US

(21) Appl. No.: 10/132,695

(22) Filed: Apr. 26, 2002

Related U.S. Application Data
(60) Provisional application No. 60/288,084, filed on May 3, 2001.

(51) Int. Cl. C12N 5/08

(52) U.S. Cl. 435/372

(57) ABSTRACT

Purification of rare Hematopoietic Stem Cell(s) (HSC) to homogeneity is required to study their self-renewal, differentiation, phenotype, and homing. Long-term repopulation (LTR) of irradiated hosts and serial transplantation to secondary hosts are the gold standard for demonstrating self renewal and differentiation, the defining properties of HSC. We show that rare cells that home to bone marrow can LTR primary and secondary recipients. During the homing, CD34 and SCA-1 expression increases uniquely on cells that home to marrow. These adult bone marrow cells have tremendous differentiative capacity as they can also differentiate into epithelial cells of the liver, lung, GI tract, and skin. This finding may contribute to clinical treatment of genetic disease or tissue repair.

This invention was made using funds from the U.S. government under grants from the National Institutes of Health numbered RO1HL 54330, RO1DK 53812, P01CA 70970. The U.S. government therefore retains certain rights in the invention.

### BACKGROUND OF THE INVENTION

Several recent reports suggest that there is far more plasticity than previously believed in the developmental potential of many different adult cell types. Recently, we and others showed that a bone marrow population enriched for HSC can differentiate into mature hepatocytes in the liver of rodents (Petersen et al., 1999; Theise et al., 2000a), and this differentiation of bone marrow cells into mature cells of the liver, also occurs in humans (Theise et al., 2000b; Alison et al., 2000). Other examples of this surprising plasticity include the in vivo regeneration of murine skeletal muscle cells from bone marrow cells (Ferrari et al., 1998) and of bone marrow from skeletal muscle cells (Jackson et al., 1999). Some of these studies have shown mesodermally derived tissue arising from ectodermally derived tissue and vice versa, such as the reconstitution of bone marrow from cultured brain (Bjornson et al., 1999) and glial cells arising from bone marrow (Eglitis and Mezey, 1997). Therefore, the boundaries determined by embryologic trilaminar origin are not maintained in the adult. The phenotype of the bone marrow sub-population that has this increased plasticity is not yet known. Here we study whether a unique bone marrow subpopulation highly enriched for hematopoietic stem cells also has the ability to differentiate into epithelial cells previously thought to be exclusively of endodermal derivation.

HSC are present in mouse bone marrow at a frequency of 1 in 10⁵ cells (Harrison et. al., 1990). The rarity of these cells and the absence of specific markers have made the search for a pure HSC population a challenge for the past 50 years. The lack of ideal in vitro assays for HSC requires that functional assays be utilized to establish their presence. We and others have shown that LTR is possible with small numbers (1-10) of HSC (Jones et al., 1996; Spangrude et al., 1995; Osawa et al., 1996) but serial transplantation (self-renewal) of single cell reconstituted recipients serving as donors for new recipients has not yet been shown convincingly.

There is a continuing need in the art for better methods of performing bone marrow transplantation.

### SUMMARY OF THE INVENTION

In one embodiment of the invention a homogeneous preparation of one or more mammalian hematopoietic stem cells is provided.

In another embodiment of the invention a method is provided for isolating a homogeneous preparation of hematopoietic stem cells. Bone marrow cells of a donor mammal are isolated via elutriation; cells are collected at a flow rate of 20-35 ml/min to form a fraction of bone marrow cells. The fraction of cells is depleted of lineages selected from the group consisting of: T lymphocytes, B lymphocytes, macrophages, granulocytes, erythroid cells, late progenitor cells and combinations thereof, using antibodies specific for markers of said lineages. The lineage-depleted fraction is labeled with a dye that binds to fatty acids in cell membranes. The labeled lineage-depleted fraction is injected intravenously into a lethally irradiated first mammalian recipient; the injected cells home to recipient organs for 2 days. A fraction of dye-containing cells which are as dye-bright as dye-labeled cells before said step of injecting is recovered from the first recipient's marrow via flow cytometry and cells which are 6-8 μm in diameter by forward light scattering are collected. A homogeneous preparation of hematopoietic stem cells is thereby formed.

In still another embodiment of the invention another method for isolating a homogeneous preparation of hematopoietic stem cells is provided. Bone marrow cells of a donor mammal are fractionated via elutriation and cells are collected at a flow rate of 20-35 ml/min to form a fraction of bone marrow cells. The fraction of cells is depleted of lineages selected from the group consisting of: T lymphocytes, B lymphocytes, macrophages, granulocytes, erythroid cells, late progenitor cells and combinations thereof. The depletion is accomplished using antibodies specific for markers of said lineages. The lineage-depleted fraction is labeled with a dye which binds to fatty acids in cell membranes. The labeled, lineage-depleted fraction is cultured on an irradiated stromal cell culture for 2 days. A fraction of dye-containing cells which are as dye-bright as dye-labeled cells before said step of culturing is recovered via flow cytometry of the cultured, labeled, lineage-depleted fraction, and cells which are 6-8 μm in diameter by forward light scattering are selected to form a homogeneous preparation of hematopoietic stem cells.

### BRIEF DESCRIPTION OF THE DRAWINGS

FIGS. 1A and 1B show immunohistochemical and FISH analysis of bronchus. Light microscopic image (orig. mag. 20×) of bronchus stained by immunohistochemistry using antibody Cam5.2, specific for cytokeratins 8, 18, and 19. Epithelial cells are positive with dim cytoplasmic and dark membranous staining. Other cells are negative. Cells are counterstained with hematoxylin. The arrows indicate Y-chromosome positive epithelial cells. The arrowhead on the right indicates a Y-chromosome positive cell that does not express cytokeratins recognized by Cam5.2 and is located below the epithelium within the lamina propria; it is therefore probably either a stromal cell or a cell of hematopoietic lineage.

FIG. 1C shows fluorescence microscopic image (100×) of FISH for Y chromosome (pseudocolored pale yellow green), with DAPI (blue) nuclear counterstain. This image is from the same slide as in FIGS. 1A and 1B. Morphology of cells and persistence of DAB stain indicating cytoplasmic cytokeratins define the bronchial epithelial lining cells. Yellow Y-chromosomes are identified in three such cells (arrows). The submucosal collagen autofluoresces and is pseudo-colored green using a combination of filters (Cy5 for DAB, Cy3.5 for rhodamine, FITC for autofluorescence, DAPI for nuclei).

FIG. 2A shows immunohistochemical and FISH analysis of small intestine. Light microscopic image (100×) of a cross-section of a small intestinal villous showing double immunohistochemical staining with anti-cytokeratin antibody CAM5.2, specific for epithelium (brown), and with anti-CD11b antibody Mac1, specific for macrophages (red). Macrophages are confined to the lamina propria, are not found above the basement membrane within the epithelial surface, and do not co-express cytokeratins. Cytokeratin positive epithelial cells never co-express CD11b. (DAB, Fuchsin-Red, Mayer's hematoxylin). FIG. 2B shows fluorescence microscopic image (100×) of a small intestinal villous from the same double immunostained slide after FISH for Y chromosome (red) and DAPI (blue) nuclear counterstain. Morphology of cells and persistence of DAB stain indicating cytoplasmic cytokeratins define the intestinal epithelial lining cells, two of which (right middle) display red Y-chromosomes. No CD11b-positive macrophages were seen in this particular cross-section. (Filters: Cy5, Cy3.5 for rhodamine, FITC for autofluorescence, DAPI for nuclei).

FIGS. 3A-3F show epithelial lining cells of the lung alveoli, GI tract, cholangiocytes, and hair follicle cells show male marrow-derived derivation. Fluorescence microscopic images of FIG. 3A. lung, FIG. 3B. esophagus, FIG. 3C. stomach, FIG. 3D. colon, FIG. 3E bile duct cyst, FIG. 3F. skin. (Filters as above, original magnifications of FIG. 3A, FIG. 3C, and FIG. 3E 100×, FIG. 3B, FIG. 3D, and FIG. 3F, 60×). Due to pseudo-coloring of images to enhance cellular details, Y chromosomes appear yellow in A, and blue-green in FIG. 3B-FIG. 3F.

FIGS. 4A and 4B show double FISH for surfactant B mRNA and Y chromosome confirms identity of donor derived epithelial cells in the lung. The upper image (FIG. 4A) was obtained by overlaying the fluorescence image obtained with the DAPI (blue nuclei), FITC (green transcription centers) and Cy3.5 (red Y chromosome) filters. The lower image (FIG. 4B) was obtained by using the “find edges” command in Adobe Photoshop after increasing the gain to detect the autofluorescence of the cell bodies (shown black in this schematic).

### DETAILED DESCRIPTION OF THE DRAWINGS

It is a discovery of the present inventors that a homogeneous preparation comprising one or more mammalian hematopoietic stem cells can be isolated. The preparation does not cause Graft vs. Host Disease upon transplantation. The hematopoietic stem cells in the preparation are 6-8 μm in diameter as measured by forward light scattering. The preparation is capable of self-renewal and reconstituting all bone marrow derived cells upon serial bone marrow transplantation, even if the preparation comprises just a single cell.

The homogeneous preparation of hematopoietic stem cells can be made in at least one of two ways. In one method a homing step is employed, during which the preparation of cells is briefly passaged through a recipient mammal. Cells which go to the bone marrow compartment are isolated after 2 days. These cells are found to be remarkably enriched for engraftment ability. According to an alternative method cells are cultured on a culture of irradiated stromal cells. After 2 days of in vitro “homing” cells are isolated as described and again have an enriched engraftment ability. The methods are described in more detail below.

The first step in either method employs a fractionation of bone marrow cells of a donor mammal via elutriation. Elutriation is well known in the art and is described in Jones, U.S. Pat. No. 5,876,956, Noga, “Engineering hematopoietic grafts using elutriation and positive cell selection to reduce GVHD,” Cancer Treat. Res. 101:311-30, 1999, Gengozian et al., “Relative sedimentation of hematopoietic progenitors in human cord blood, peripheral blood, and bone marrow as determined by counterflow centrifugal elutriation,” Transplantation 65: 939-46, 1998, Inoue et al., “Separation and concentration of murine hematopoietic stem cells (CFUS) using a combination of density gradient sedimentation and counterflow centrifugal elutriation,” Exp. Hematol. 9: 563-72, 1981. Fractions of cells are collected from the elutriation at flow rates of 20-35 ml/min. Preferably the flow rate is 23-28 ml/min, and more preferably it is 25 ml/min. Subsequently the fraction of cells is lineage depleted. This is an immunological technique whereby antibodies which are specific for specific differentiated hematopoietic cell lineage markers are used to bind to cells, and subsequently to remove such cells from the fraction. Many antibodies which are specific for markers which are characteristic of such differentiated cell lineages are known in the art and any of these antibodies can be used. The cell fraction is desirably depleted by this immunological method of lineages including T lymphocytes, B lymphocytes, macrophages, granulocytes, erythroid cells, late progenitor cells and combinations thereof. Susequently the lineage-depleted fraction is labeled with a dye which binds to fatty acids in cell membranes. Any such dye may be ued including PKH26 and CFSE, although the former is preferred because it labels cells faster than the latter. PKH26 (Sigma) is an aliphatic reporter molecule which incorporates into the cell membrane lipid bilayer. PKH26 is a red fluorochrome, having an excitation (551 nm) and emission (567 nm) compatible with rhodamine or phycoerythrin detection systems. Other PKH dyes are green fluorochromes and can also be used.

The labeled lineage-depleted fraction is then passaged though a lethally irradiated mammalian recipient. Typically the cells are injected intravenously, but other modes of administration, such as intraperitoneal and subcutaneous injections can be used. Such modes, however, are believed to be less efficient for engraftment. The cells in the labeled lineage-depleted fraction are permitted to “home” to recipient organs for 2 days. After 2 days the injected cells can be found in many different organs. However, cells are desirably recovered from the recipient's bone marrow. The recovered cells are subjected to flow cytometry. Cells are selected on two bases: forward light scatter for size and staining for the dye. The gate for dye intensity is strictly set so that the cells which are selected are of the same intensity of dye staining as those cells which were stained prior to transplant. In addition, cells are selected as being 6-8 μm in diameter as measured by forward light scattering. The cells thus passaged and selected form a homogeneous preparation of hematopoietic stem cells. Unlike prior methods of enriching for hematopoietic stem cells, the present method does not employ a step of selecting cells for expression of aldehyde dehydryogenase.

Mammals which can be used to passage the cells for the homing step can be of any species, including cows, sheep, dogs, cats, human, rats, mice. Preferred mammals are immunodeficient animals, irradiated animals, and pre-immune fetuses of mammals. Particularly preferred are immunodeficient mice and pre-immune fetal sheep.

The isolated cells can be diluted to the limit, so that each sample comprises just one cell. The presence of single viable cells in diluted samples can be confirmed by direct observation. The use of single cells for transplantation demonstrates that one single progenitor cell is sufficient for complete and durable engraftment.

The homogeneous preparations of the present invention can be used for transplantation into the same mammal as the cell donor or into a second mammalian recipient. Similarly the recipient in which the cells are passaged for 2 days for homing can be the same individual as or different from the cell donor and the ultimate transplant recipient.

The second method for making the homogeneous preparation of hematopoietic stem cells is performed identically to the first method in all respects but one. Rather than doing an in vivo homing step, the cells are homed in vitro. For in vitro homing labeled, lineage-depleted cells are cultured on a layer of stromal cells. After 2 days the cells are collected and selected as described in the first method. Stromal cells can be of the same or different individual mammal, or can be of the same or different species. Preferably the cells are of the same species. Stomal cells which can be used include stromal cell lines, as well as primary cultures of plastic-adherent cells obtained from whole bone marrow.

The source of the HSC cells may be the bone marrow, fetal, neonate, or adult or other hematopoietic cell source, e.g., fetal liver or blood. For example, antibodies linked to magnetic beads may be used initially to remove large numbers of lineage committed cells, namely major cell populations of the hematopoietic systems, including such lineages as T cells, B cells (both pre-B and B cells), myelomonocytic cells, or minor cell populations, such as megakaryocytes, mast cells, eosinophils and basophils. Preferably, at least about 70%, usually at least 80%, of the total hematopoietic cells will be removed. It is not essential to remove every dedicated cell class, particularly the minor population members at the initial stage. Usually, however, the platelets and erythrocytes will be removed prior to fluorescence sorting. Since there will be positive selection in the protocol, the dedicated cells lacking the positively selected marker will be left behind. However, it is preferable that negative selection is done for all of the dedicated cell lineages, so that in the final positive selection, the number of dedicated cells present is minimized.

The methods of this invention have therapeutic utility. For instance, the homogeneous preparation of hematopoietic stem cells (HSC) and/or pluripotent HSC obtained by the method of this invention can be used for performing hematopoietic reconstitution of a recipient using a preparation derived from the recipient (autologous reconstitution) or derived from an individual other than the recipient (non-autologous reconstitution) in the treatment or prevention of various diseases or disorders such as anemias, malignancies, autoimmune disorders, and various immune dysfunctions and deficiencies, as well as recipients whose hematopoietic cellular repertoire has been depleted, such as recipients treated with various chemotherapeutic or radiologic agents, or recipients with AIDS. Other therapeutic uses of the compositions of the invention are well known to those of skill in the art.

The method of providing a homogeneous composition of hematopoietic stem cells (pluripotent HSC) can be used to separate the progenitor stem cells useful in a bone marrow transplant from the other healthy cells in a bone marrow aspirate provided by a healthy bone marrow donor. Alternatively, when the donor is the patient, the method of this invention can be used prior to treatment of the patient with chemotherapeutic or radiologic agents, to separate the pluripotent HSCs for eventual reintroduction into the patient after therapy has been completed. In the latter case, the cancer cells can be removed from the cell mixture comprising the bone marrow aspirate by tagging the cancer cells with characteristing markers, such as cancer-specific cell surfaces markers. One skilled in the art will appreciate that alternative methods well known in the art, such as ex vivo magnetic cell sorting, can also be employed to remove cancer cells from the cell mixture before subjecting the cell mixture to cell sorting.

The human stem cells provided herein find a number of uses, for instance: (1) in regenerating the hematopoietic system of a host deficient in stem cells; (2) in treatment of a host that is diseased and can be treated by removal of bone marrow, isolation of stem cells, and treatment of the host with therapeutic agents such as drugs or irradiation prior to re-engraftment of stem cells; (3) as a progenitor cell population for producing various hematopoietic cells; (4) in detecting and evaluating growth factors relevant to stem cell self-regeneration; and (5) in the development of hematopoietic cell lineages and screening for factors associated with hematopoietic development.

Our studies show that bone marrow populations can be enriched significantly for stem cells by recovering cells that home to the bone marrow within 48 hours of transplantation. This purifies functional HSC from one in 10³ Fr25lin⁻ marrow cells to approximately one in six (16.6% of mice engrafted at 11 months). Significantly, single bone marrow derived cells have non-hematopoietic differentiation potential as well. This level of enrichment may be an underestimate because, as has been suggested (Osawa et. al., 1996), only 20% of recipients are likely to receive the single cell in a marrow niche (seeding efficiency), the site best suited for expansion and self renewal of HSC. Initial experiments were performed using limiting dilution to transplant 1 male derived cell per recipient. The percentage of animals that received greater than one cell calculated by Poisson statistics and viability (50% to 60% viable by propidium iodide staining) was no greater than 7-9%. The cells were elutriated, lineage depleted by anti-body treatment, labeled with PKH26, passaged in a mouse for two days and passed through the cell sorter followed by re-transplantation into new recipients all contributing to this level of viability. We are confident that since 17% of the animals engrafted, at least some of them received a single cell. We have repeated these studies using direct visualization of single viable cells, rather than depending upon limiting dilution prior to injection into mice for long-term engraftment. Fifty six percent of these mice are alive at three months. We observe both hematopoietic and non-hematopoietic (epithelial) engraftment in a sample of these mice which further supports the conclusion that multi-organ multi-lineage engraftment occurred.

The high level of engraftment of blood and marrow 11 months post transplant suggests that the expansion and differentiation of a single marrow SC to reconstitute the majority of the hematopoietic system of a lethally irradiated recipient is feasible. Three of the long term survivors had greater than 70% donor cells in the blood at 5 months post transplant and 50% or more male cells at 11 months. This is also reflected in the large number of donor-derived progenitor cells (CFU) at 11 months (greater than 70%). Two of the mice appear to have lost most of their graft (mice #1 and #5), but only mouse #1 also lost donor type colony forming progenitors at 11 months. Even the mouse with little peripheral blood engraftment and no marrow progenitor engraftment at 11 months (Mouse #1) had non-hematopoietic cell engraftment at this time.

The variable level of engraftment following single cell transplantation is likely due to donor HSC dilution (Jones et al., 1989) in the recovering host and the variations in successful homing to the marrow space, which is necessary for successful seeding of HSC. Adhesion molecules required for homing include VLA-4 (Craddock et. al., 1997). Since CD34 may also have a role in adhesion (Healy et. al., 1995) it is intriguing to speculate that up-regulation of CD34 in donor cells is required for engraftment and may be related to marrow homing. Alternatively, CD34+ donor cells have a homing advantage. Recent studies suggest reversible expression of CD34 both in-vivo (Sato et. al., 1999) and in-vitro (Sato et. al., 1999; Nakamura et. al., 1999) due either to cytokine stimulation or cell cycle activation following 5 FU administration. In contrast to these data in which changes in CD34 expression occur much later, we may be observing an increase of CD34 within the first 48 hours post transplant. At two days our cells are not in cell cycle (Lanzkron et.al., 1999) as would be the case with 5FU or cytokine exposure. It is possible that HSC require CD34 expression to maintain LTR potential and home to the marrow or that up-regulation of CD34 expression occurs soon after cells arrive in the marrow space. In any case, the change from 4% to 45% CD34 positive cells is a reflection of an early step which may be necessary for our single HSC to LTR recipient mice.

Donor derived epithelial cells were detected in lung, GI tract, and skin, and were distinguished from intraepithelial hematopoietic cells (i.e. lymphocytes, polymorphonuclear leukocytes, and macrophages) by their cytokeratin staining, morphology, and examination of parallel sections. The cytokeratins detected by the monoclonal antibodies employed here are specific for epithelial cells and are not identified in cells of any hematopoietic lineage (Moll, et al. 1982, Sun, et.al. 1984). Moreover, when double immunohistochemistry was performed with anti-macrophage specific antibodies, single cell co-localization of the two markers never occurred, confirming that these cytokeratin positive cells were not macrophages that had phagocytosed debris of dead epithelia.

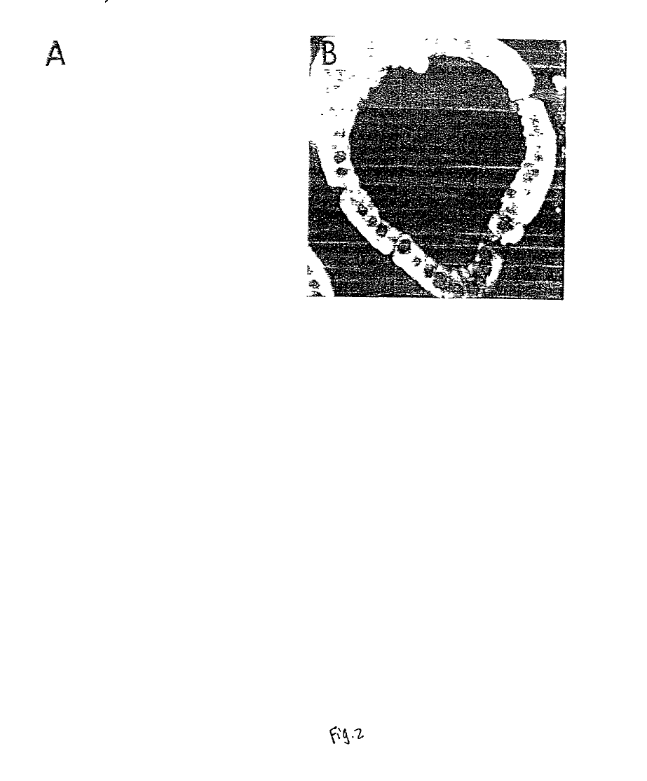

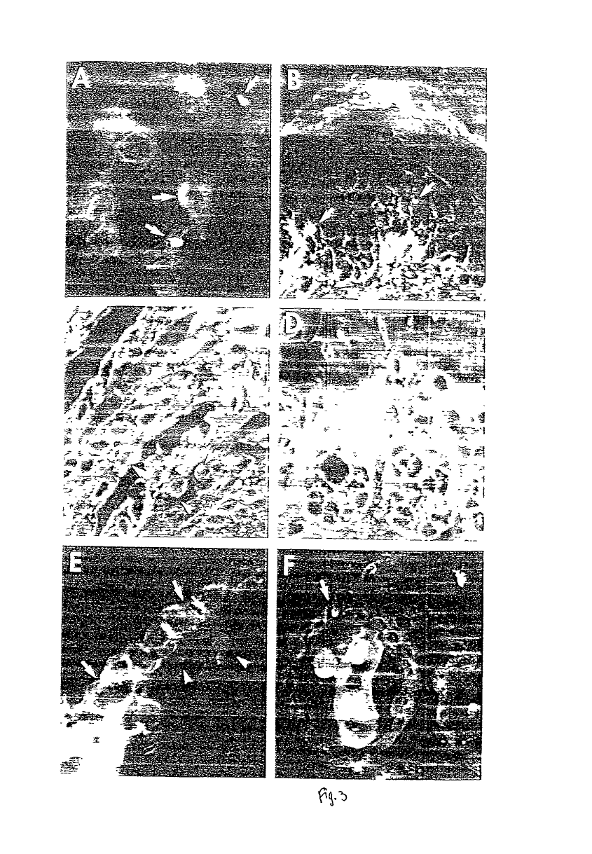

The epithelial engraftment was found at different frequencies in different organs. These differences may be due to 1) the degree of tissue damage induced by the transplant, 2) the residual tissue-specific stem cell capacity within each organ, and/or 3) the normal rate of cell turnover in each organ. These possibilities are supported by the variable levels reported for liver engraftment by marrow derived cells. With injury or genetic deficiency sufficient to evoke an intrahepatic stem cell proliferation, clusters of marrow-derived hepatocytes, cholangiocytes, and oval cells form (Petersen et. al., 1999; Lagasse et. al., 2000). In the absence of such injury (Theise et. al., 2000a; Theise et. al., 2000b), isolated, scattered hepatocytes and cholangiocytes develop, suggesting that they engraft in the liver in what appears to be a random process which may bypass an intrahepatic stem cell intermediate. At the time of analysis (11 months post-transplant) no histological evidence of damage was apparent in any of the tissues examined. Clusters of Y chromosome positive cells were detected only in alveolar lining cells (FIG. 3A). The high levels of donor engraftment as lung cells are analogous to those seen in severe injury models reported for the liver (Petersen et. al., 1999; Lagasse et.al., 2000). Lung tissue is significantly damaged by radiation yielding necrosis of alveolar lining cells, focal hemorrhage and eventual scarring (Travis et.al., 1985). Alternatively, there may be lung tissue damage due to low level viral infection in these temporarily immunosuppressed animals. In mice examined within the first week following lethal irradiation, there is focal hemorrhage and macrophage infiltration within the lung parenchyma (authors' unpublished data). Within this damaged lung tissue, surfactant B producing (type II) pneumocytes engrafting from transplanted marrow were detected as early as 5 days post-transplant (unpublished data). Type II pneumocytes are thought to be the alveolar progenitor cells, giving rise to type I pneumocytes in response to injury (Magdaleno et al., 1998). Both of these pneumocyte populations can be demonstrated by immunostaining for the same panel of cytokeratins; thus the cells pictured in FIG. 2 represent a mixture of type I and II alveolar lining cells. Therefore, the high percentage of Y chromosome positive pneumocytes may reflect an early proliferative healing response to acute radiation injury and possibly to post-radiation infection.

Thus there are two patterns of epithelial engraftment of marrow derived cells: large-scale repopulation in response to injury (as demonstrated in liver and lung) and low level engraftment as individual scattered cells in the absence of marked injury (e.g. liver, skin, and GI tract). These randomly inserted single cells may not be fully functional since they do not appear to proliferate.

The data presented herein demonstrate a high degree of plasticity with a single cell having the ability to differentiate into cells of the GI tract, lung, and skin. Although little is known about how these cells obtain this degree of differentiative potential, it is possible that the cells are “summoned” to sites of injury by factors secreted from the damaged organ. Once the cells arrive in the damaged tissue, the local environment stimulates gene expression patterns that cause a morphological change in the phenotype of the cell. Interestingly, theories regarding how cells undergo cell type specific differentiation strongly suggest that tissue specific transcription factors are rare. Rather, different combinations of the same transcription factors present in different ratios induce different patterns of gene expression that cause cells to differentiate down different pathways (Rosen et al., 1998; Shivdasani and Orkin, 1996; Sieweke and Graf, 1998; Zahnow et al., 1997).

We conclude that passage of a partially purified marrow SC population for two days in a lethally irradiated recipient results in enrichment of cells with the capacity to LTR mice. Single bone marrow cells can self renew in vivo as well as differentiate into hematopoietic progenitors and mature cell types of both hematopoietic and non-hematopoietic tissues. Expression of CD34 is increased in mice shortly after transplantation in the marrow consistent with this molecule being involved in homing.

There are multiple therapeutic implications of this work. Bone marrow derived cells that have the capacity to differentiate into mature epithelial cells can serve as target cells for gene therapy or as a source for organ reconstitution and repair. Bone marrow transplantation itself is useful in the treatment of some forms of tissue injury or disease. For example, gene therapy for various pulmonary disorders will require infection of a stable and renewing population of cells with expression of the desired gene product under normal physiologic control (e.g. Cystic Fibrosis Transmembrane Regulator). Pneumocytes would be an excellent target for gene therapy. One could design gene therapy vectors on which drugs that can inactivate viruses are expressed only in virus infected cells. Populations of bone marrow stem and progenitor cells can be infected with high efficiency by retroviral vectors (Abonour et.al., 2000; Ito and Kedes, 1997; Nolta et.al., 1992; Nolta et.al., 1996) making the bone marrow SC a potential delivery system for hematological and epithelial gene therapy.

### EXAMPLES

### Example 1

This example discusses the rationale the purification of a pure population of HSC.

Using a two-day homing protocol we tested whether individual marrow cells that rapidly home to the bone marrow are enriched for HSC. We use a membrane bound dye (PKH26; Sigma) to track and recover cells from specific locations in-vivo. This allows us to determine cell cycle activity as the dye is equally distributed to each daughter cell. We demonstrate that at least some HSC home to the bone marrow and remain quiescent for up to 48 hours following transplantation. After labeling and injection into a first female recipient, quiescent male cells that are recovered from the bone marrow 48 hours post transplantation are capable of LTR when transplanted into other female mice (Lanzkron et al., 1999). In the current study our goals were to see if these recovered cells are enriched for a pure population of HSC with LTR ability and to examine the potential of limited numbers of these bone marrow-derived stem cells to engraft non-hematopoietic tissues.

It is not yet known which bone marrow cells are capable of differentiation. Based on previous data by which we showed that purified CD34+lin− bone marrow cells can differentiate into hepatocytes in the liver (Theise et al., 2000a), we hypothesize that the same cells that reconstitute hematopoiesis can also differentiate into non-hematopoietic tissues. We test this by examining the non-hematopoietic tissues of animals that engraft with functionally isolated (homed) bone marrow cells.

Many different surface markers have been used to identify and isolate HSC from mouse bone marrow, and a consensus regarding which markers are consistently expressed on these cells has not yet been reached. An emerging body of work suggests that the HSC may not display CD34 (Goodell et. al., 1996; Zanjani et. al., 1998; Bhatia et. al., 1998), as was previously thought (Krause et. al., 1994; Morel et. Al., 1996). Osawa, et. al. (1996) showed that a single HSC expressing a low level of CD34 message could LTR mice and our group has shown low expression of CD34 on HSC (Jones et. al., 1996). We have subsequently demonstrated (Donnelly et. al., 1999) that the HSC compartment is phenotypically heterogeneous with populations of HSC that are positive and negative for CD34 expression. It may be that expression of CD34 is related to cell cycle activation (Sato et. al., 1999) and may be reversible in-vitro (Nakamura et. al., 1999). The homing assay used herein enriches for HSC without using specific surface markers to identify the cells. We have analyzed the expression of CD34 and SCA-1 on these cells before and after they home to the marrow and spleen.

### Example 2

This example demonstrates hematopoietic engraftment and self-renewal.

Male donor marrow cells, first fractionated (Fr 25) via elutriation, and then lineage depleted (lin−), were labeled with PKH26, and injected intravenously into lethally irradiated female recipients as we previously described (Lanzkron et al., 1999). Two days post transplant, PKH26 bright donor cells were recovered by flow cytometric sorting of recipient bone marrow. By limiting dilution, 30 new irradiated female hosts were each transplanted with a single recovered PKH26 labeled cell. Survival and donor reconstitution were assessed for 11 months post transplant. We previously demonstrated that 10² cells that homed to marrow, but not 10⁴ cells that homed to spleen, had LTR ability (Lanzkron et al., 1999). In our current study, as a control, 10³ or 10² PKH26+ FR25Lin− cells from male donors were transplanted into female recipients for LTR without first utilizing the homing procedure. The control animals did not survive past twelve weeks or had no male donor cell reconstitution prior to death (data not shown). Of the 30 mice transplanted with a single recovered PKH26 bright cell, 5 survived long-term. In Table 1, the percent donor cell reconstitution is shown for the surviving recipients 5 and 11 months post transplant. Because 17% of animals that received a single male cell showed long-term male reconstitution, there is a 500-1000 fold enrichment of LTR cells after homing of the Fr25lin− starting population.

TABLE 1
Engraftment and self-renewal potential of a single HSC
transplanted into lethally irradiated mice.

| | % Donor Cells | | Percent | Engraftment | |
|---|---|---|---|---|---|
| | (Peripheral | | Male | after Serial | |
| | Blood) | | CFU* | Transplantation§ | |
| | 5 mo | 11 mo | 11 mo BM | 2 mo PB | 4 mo PB |
| Mouse 1 | 30 | 13 | 0.0 | 0 | 1 ± 0 |
| Mouse 2 | 76.5 | 54.5 | 77.5 | 15 ± 04 | 49 ± 0.4 |
| Mouse 3 | 91 | 75.5 | 95.5 | 18 ± 0.1 | 38 ± 0.2 |
| Mouse 4 | 85.5 | 86.5 | 97.5 | 28 ± 0.10 | 77 ± 0.1 |
| Mouse 5 | 78 | 12 | 88.0 | 1 ± 0 | 2.5 ± 0 |

*FISH was performed on pooled myeloid and erythroid progenitors harvested from each individual mouse.
§Peripheral blood (PB) was drawn from secondary recipients at 2 and 4 months post-transplant. Shown are the mean + SE for the 4 recipients of donor marrow from each of the 5 long-term surviving mice.
BM = bone marrow.

The five long-term survivors of a single cell were sacrificed at 11 months and cells from each of their marrows were plated for hematopoietic progenitors and also used for serial transplantation. Table 1 shows that marrow from four of the five survivors had between 77.5 and 97.5% male derived colonies. Mouse #1 which only had 13% donor peripheral blood cells had no detectable male donor progenitor cell activity at 11 months post transplant but mouse #5 which also had low peripheral blood donor cells (12%) had 88% donor derived colonies. The engraftment (two and four months after serial transfer of 10⁶ cells from each of the 5 primary long-term survivors) into groups of four new female lethally irradiated recipients is shown in Table 1. Mice #2, 3, and 4 provided marrow that engrafted recipients with male cells four months post serial transplant approaching a level of engraftment equal to that observed in the primary recipient. This represents strong evidence for HSC self-renewal.

### Example 3

This example shows cell-surface antigen expression of HSC.

We examined the frequency and absolute number of CD34 and SCA-1 positive cells labeled with PKH26 prior to and 48 hours after transplantation into lethally irradiated recipient mice. Recovered PKH26 bright (quiescent) cells from the bone marrow of the recipients had higher frequencies of CD34+ and SCA1+ cells (46% and 24%, respectively) compared with the starting population (approximately 4% and 3% CD34+ and SCA1+ cells, respectively, Table 2). It is not clear whether the cells that homed to the marrow underwent an up-regulation of CD34 expression, or if CD34 expressing cells from the starting population homed preferentially to the marrow. If the latter possibility is true, then 29.5% and 9.3% of the CD34+ and SCA1+ injected cells, respectively, home to the marrow as opposed to 12.75% of the total cell population which we reported previously (Lanzkron et al., 1999). In contrast, PKH26 bright cells recovered from the spleen after 48 hours were not enriched for CD34+ and SCA1+ cells.

TABLE 2
The FR25 Lin- PKH+ CD34+ and Sca-1+ frequency before and
and after transplant and the absolute recovery 48 hrs
post transplantation.

| | | DAY 0 | | |
|---|---|---|---|---|
| ORGAN | PHENOTYPE | (% +) | DAY 2 (% +) | % REC.* |
| BM | CD34 | 4.2 ± 0.01 | 45.8 ± 0.16 | 29.5 ± 6.33 |
| NM | SCA-1 | 3.4 ± 0.02 | 24.8 ± 0.10 | 9.3 ± 4.40 |
| SPL | CD34 | N/A | 9.4 ± 0.04 | 4.8 ± 2.10 |
| SPL | SCA-1 | N/A | 7.0 ± 0.03 | 3.3 ± 0.63 |

Values represent the mean ± SEM for three experiments.
*The absolute % recovery is calculated as the total number of Fr25Lin-
PKH+ CD34+ or SCA-1+ cells in the bone marrow or spleen
at 48 hrs divided by the number of CD34+ and SCA+ cells
injected. The total number of marrow cells is determined by dividing
the number of cells in the two hind limbs by 16%, the percentage
of the total skeletal marrow that these bones represent.
$Absolute \% recovery = \frac{\begin{matrix}\# of PKH \text{+} cells recovered \times \\ \% antigen positive cells recovered\end{matrix}}{\begin{matrix}\# of PKH \text{+} cells injected \times \\ \% antigen positive cells injected\end{matrix}}$
The Frequency measurements are the % positive cells for both PKH
and CD34 or PKH and SCA-1 with a total of 10⁴ cells examined.
N/A = not applicable

### Example 4

This example demonstrates stem cell homing in CD34 knockout mice.

To further examine the role of CD34 in stem cell homing we used the 2 day homing assay to assess localization of cells from CD34 knockout mice in the spleen. PKH26+ Fr25Lin− cells from mice with a disruption in their CD34 gene (Suzuki et al., 1996) seeded the spleen of normal recipient mice to a greater extent than did normal HSC (data not shown). This finding provides additional evidence that CD34 may be responsible in part for the directed homing of cells with LTR ability early after transplant.

### Example 5

This example demosntrates engraftment of epithelial tissues in long-term chimeric mice.

Analysis of the epithelial tissues from the 5 mice that had been transplanted with single “homed” cells yielded a surprisingly extensive differentiation repertoire. Immunostaining for cytokeratins was used to identify epithelial cells in the tissues. The staining pattern of the cytokeratins in multiple organs is indicated in Table 3.

TABLE 3
Summary of Immunohistochemical Staining

| | Anti-cytokeratin | |
|---|---|---|
| | Monoclonal Antibody | |
| | AE1/AE3 | Cam5.2 |
| Epithelial cells of: | | |
| Stomach | ++ | + |
| Esophagus | + | ++ |
| Small intestine | ++ | ++ |
| Large intestine | ++ | ++ |
| Liver | | |
| Cholangiocytes | ++ | + |
| Hepatocytes | 0 | 0 |
| Kidney | | |
| Glomeruli | 0 | 0 |
| Tubules | 0 | + |
| Lung | | |
| Bronchi | ++ | + |
| Pneumocytes | 0 | ++ |
| Skin | + | ++ |

Staining: 0 Absent; + variable; ++ diffuse, strong.

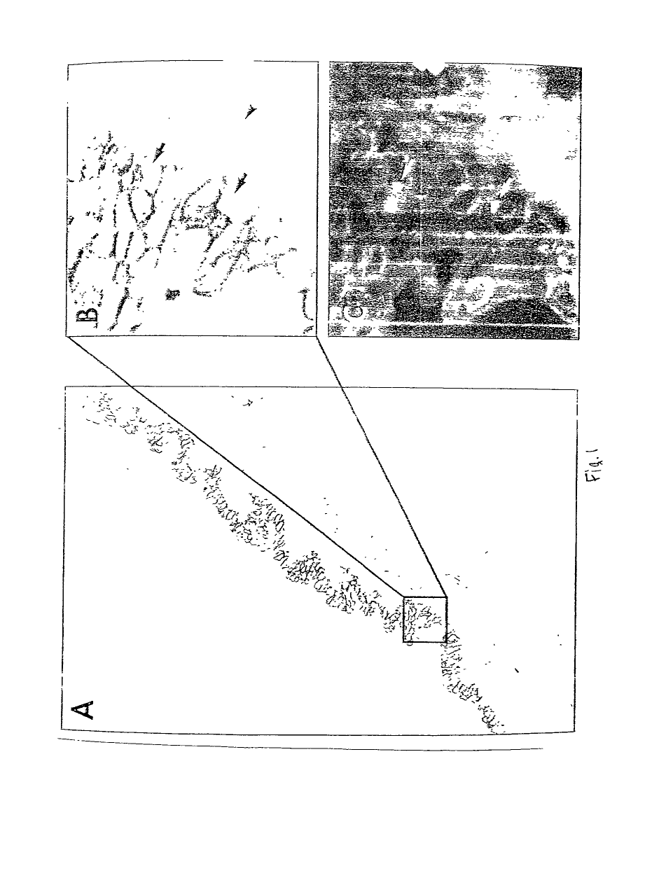

Quantitative analysis of donor cell reconstitution was performed only for those cell types that could be definitively identified by these antibodies. Based on the data presented in Table 3, therefore, the tissues examined included lung (bronchi and alveoli), esophagus, stomach, small bowel, colon, renal tubules, biliary tree (cholangiocytes), and skin. Y chromosome positive cells developed in the bronchi as shown in FIG. 1. In this figure, the double staining approach is shown in detail. FIG. 1A shows a representative low power light microscopic image. The columnar respiratory epithelium is brown due to immunoperoxidase staining with Cam5.2 antibody against cytokeratins 8, 18, and 19. A small region of this photo is reproduced larger in FIG. 1B so that single cells are apparent. FISH for the Y chromosome is shown in FIG. 1C for the identical cells as in FIG. 1B. Male, donor-derived epithelial cells lining the bronchus are identified by the two arrows on the left.

Throughout the study, intraepithelial lymphocytes were excluded as a possible source of false positive identification of epithelial cells. Examination of sequential sections of liver, lung, skin, and esophagus failed to demonstrate the presence of such lymphocytes in the regions studied by FISH (data not shown). In contrast, intraepithelial lymphocytes were present in stomach, small intestine and large intestine. Lymphocytes could be confidently excluded from our identification of epithelial cells by reliance on strict criteria for the characterization of epithelial cells. These include cell size (nuclei at least twice as large as normal lymphocytes), cytokeratin immunohistochemical staining up to the nuclear membrane, and lack of the halo indicative of lymphocyte cytoplasm (data not shown).

Macrophages were excluded as false positive cells using dual color immunohistochemical staining for the relevant cytokeratins and a macrophage-specific antibody CD11b (FIG. 2A). As shown in this cross-section through a villous of the small intestine, cytokeratins (stained brown with DAB) and CD11b (stained red with fuchsin red) do not co-localize. While numerous macrophages could be identified in the lamina propria underlying the epithelia-lined surfaces, no intraepithelial macrophages were ever identified by this double staining technique. Analysis of engraftment of small bowel epithelial cells is shown in FIG. 2B. In this cross-section through a villous of the small bowel that has been stained by FISH for the Y chromosome, 2 adjacent Y chromosome positive epithelial cells can be seen on the right. These cells clearly are located within the columnar epithelium of the small bowel, which does not contain macrophages; they have an orange autofluorescence secondary to residual DAB from the immunohistochemistry for cytokeratins, and they have the same large oval-shaped nuclei as the other epithelial cells of the villous.

Male bone marrow donor derived pneumocytes are shown in FIG. 3A. Only the fluorescence image is shown for the tissues in FIG. 3. However, the immunoperoxidase DAB staining is apparent as a red to orange to brown “pseudocolored” hue in the cell membrane and surrounding the nucleus in the cytoplasm of the epithelial cells (Pazouki et al., 1996; Theise et al., 2000b; Oosterwijk et al., 1998). In all images of FIG. 3, arrows indicate Y chromosome positive, reddish brown DAB-stained, epithelial cells. Due to partial nuclear sampling, as the plane of each 3 micron section does not always cut through the Y chromosome, Y chromosomes were visualized clearly in 62% of alveolar nuclei in a male mouse (data not shown). No Y chromosome signal was observed in female mouse tissue (data not shown). In contrast, the average number of Y chromosome positive nuclei in alveoli from the transplanted mice was 12.58±4% of epithelial cells (FIG. 3A). After correction for sampling (62% positive in male control), the mean number of male-derived alveolar cells is 20% (Table 4).

TABLE 4
Percent Donor Engraftment of Non-hematopoietic Tissues 11 Months Post-transplant

| | bronchi | alveoli | esoph | stomach | sm bowel | large bowel | skin | bile duct |
|---|---|---|---|---|---|---|---|---|
| M 1 | 3.6 | 14.8 | 0 | 0.5 | 0.3 | 0.2 | 2.6 | 0.4 |
| M 2 | 2.3 | 10.3 | 0.4 | 0.5 | 0.4 | 0.1 | 2.4 | 0 |
| M 3 | 3.5 | 18.7 | 2.2 | 0 | 0 | 0 | 1.2 | 0 |
| M 4 | 2.2 | 10.1 | 2.5 | 0.2 | 0.4 | 0.3 | 1.6 | 2.2 |
| M 5 | 0 | 9 | 0.5 | 0.4 | 1.6 | 0 | 2.7 | 0 |
| Mean ± | 2.32 ± | 12.58 ± | 1.12 ± | 0.32 ± | 0.54 ± | 0.12 ± | 2.1 ± | 0.52 ± |
| SD | 1.45 | 4.07 | 1.14 | 0.21 | 0.61 | 0.13 | 0.66 | 0.95 |
| Corr.* | 3.74 | 20.30 | 1.81 | 0.52 | 0.87 | 0.19 | 3.39 | 0.84 |

The numbers shown represent the percentage of cytokeratin positive cells within each tissue that was Y chromosome positive. For each tissue, over 150 cytokeratin positive cells were analyzed. For stomach, small bowel, and large bowel, 10³ to 3.4 × 10 ³ cells were counted for each mouse.
*The percentage of immunostained epithelial cells that are Y-chromosome positive is corrected for the male control for each tissue.

In addition to engraftment of columnar epithelial cells in the small bowel (FIG. 2), donor derived epithelial cells were identified throughout much of the GI tract including the lining of the esophagus, stomach, and large bowel as shown in FIGS. 3B-3D. In the esophagus (3B), the lamina propria is at the bottom and the lumen on the top, and the arrows indicate Y chromosome positive keratinocytes. In FIG. 3C, the branched tubular glands of the stomach are seen. The full arrow indicates a Y-chromosome positive columnar epithelial cell lining the gastric pit. The arrowheads indicate donor-derived non-epithelial cells that may be blood cells in the lamina propria. The large bowel (FIG. 3D) of each animal also had donor derived epithelial cells. In this section of colon, the donor-derived cell indicated is clearly located at the base of a gland in the mucosa of the large bowel. Importantly, additional experiments were performed in which mice were transplanted with a single visualized male bone marrow cell plus female R/O cells. These mice analyzed three months post-transplant also showed both hematopoietic and multi-organ epithelial engraftment of male cells further confirming that one cell is capable of repopulating both blood and epithelial cells.

We have shown previously that in women who were transplanted with male-derived whole bone marrow, Y chromosome positive cells comprise 4-38% of cholangiocytes after months to years (Theise, et.al., 2000b). Similarly, in the mice transplanted with Fr25Lin− homed cell, male donor-derived cholangiocytes lining the bile ducts were present. In FIG. 3E, two Y chromosome positive cholangiocytes are shown lining a biliary cyst. These DAB-stained, Y chromosome positive cholangiocytes clearly make up part of the wall of the bile cyst. Y chromosome positive cells were also present in the skin. As shown in FIG. 3F, the male donor-derived cells tended to be localized to the neck region of the hair follicles, but were also present in the epidermis (not shown). This follicular location in the neck region is a common location for the follicular “bulge,” which has recently been demonstrated to be a site for skin progenitor cells (Taylor et. al., 2000). No donor derived Y chromosome positive cells were identified amongst the cytokeratin-stained renal tubule cells of these mice.

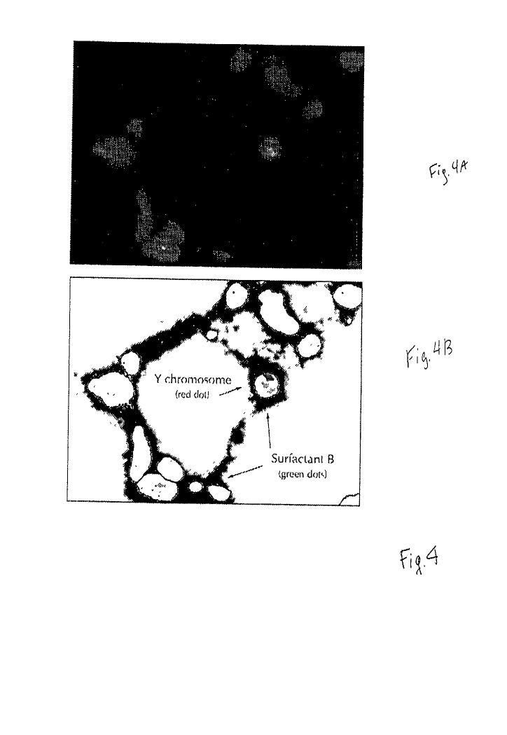

In addition to identifying epithelial cells in the organs by cytokeratin staining, we used FISH analysis for surfactant B mRNA to confirm the identity of epithelial cells in the lung. Surfactant B is transcribed exclusively in type II pneumocytes, and it is produced to such a high degree in these cells that two large transcription centers are apparent in the nuclei using a fluorescent probe for surfactant B mRNA (FIG. 4). The presence in a single nucleus of a Y chromosome and transcription centers for surfactant B identifies a male-derived type II pneumocyte. In FIG. 4, simultaneous FISH analysis for surfactant B and the Y chromosome is shown in the lung. The surfactant B transcription centers are green and the Y chromosome is red, as shown schematically in FIG. 4B.

These data not only confirm that the Y chromosome positive cells are epithelial but that they are functional cells that express tissue specific genes. Moreover, the type II pneumocyte is known to be the intraorgan stem cell in the lung parenchyma, responsible for regenerating new type II pneumocytes as well as type I pneumocytes which account for greater than 80% of the alveolar surface. Thus, engraftment of type II pneumocytes from the marrow can explain the finding. that focal alveoli were entirely lined by cytokeratin stained marrow-derived epithelia.

Quantitative analysis of donor derived cells in each of the organs examined is presented in Table 4. Significant engraftment occurred for all of the tissues examined except kidney. The highest percentage of donor engraftment (approximately 20%) occurred in the pneumocytes of the lung. The degree of engraftment throughout the GI tract was variable with the highest percent engraftment in the esophagus and the least in the colon. Although Y positive cells with the morphology and autofluorescence of hepatocytes and cardiac and skeletal myocytes were recognized, they did not stain with the anti-cytokeratin antibodies used and therefore were excluded from formal analysis in this paper.

### Example 6

This example describes the particular methodologies used in the foregoing studies.

Stem Cell Isolation and Transplantation

For bone marrow SC isolation, 20 male and female B6D2/F1 mice or male C57B1/6 CD34 knockout (kind gift from Dr. Mak, Toronto Canada) mice were killed by cervical dislocation and the hind limbs removed. Bone marrow was flushed with medium from the medullary cavities of tibias and femurs using a 25-gauge needle. Marrow cells were elutriated as previously described (Jones et. al., 1996). Male cells were collected at a flow rate of 25 ml/min (Fr25) and female cells collected after the rotor had stopped (R/O, a population enriched for progenitors and short-term repopulating cells). FR25 cells were depleted of lineage positive cells including T and B lymphocytes, macrophages, granulocytes, erythroid cells and late progenitor cell populations (FR25 Lin−) as previously described (Lanzkron et.al., 1999; Jones et. al., 1996). Male B6D2F1 or C57B1/6 knockout mice Fr25Lin− cells were labeled with PKH26 and 10⁷ labeled cells injected into lethally irradiated female B6D2F1 recipients as described (Lanzkron et.al., 1999), or in the case of the CD34 knockout experiment recipients were irradiated (1050 to 1100 cGy from a gamma cell small animal Irradiator, Atomic Energy, Canada) wildtype female C57B16/J mice. At 48 hours post transplant the female recipients were sacrificed and spleens and marrow harvested. PKH26 fluorescence intensity of single cell suspensions of spleen and marrow was measured by an Epics740 flow cytometer (Coulter Electronics, Hialeah Fla.). For transplant studies, the male FR25 Lin⁻ PKH26+ cells following passage in lethally irradiated female mice for two days were injected into additional lethally irradiated female recipients. PKH+ cells were obtained at the same intensity and size as those stained before the first transplant.

In more detail, the small cells collected by counterflow centifugal elutriation at a flow rate of 25 ml/min., 3000 rpm-1260 g (8-10 um) were further selected as only those small sized cells which were measured on the fluorescence activated cell sorter by forward light scatter as 6-8 um two days post transplant. The size was estimated by comparison to standard sized flourescent beads. Thus the cells' size was measured by forward light scatter. The cells 2 days post transplant were collected on the basis of both foward light scatter for size and stained brightly for the PKH26 dye. The gate for PKH26 dye intensity in these experiments was strictly set so that the small sized cells were of the same intensity of dye staining as those cells which were stained prior to transplant (time 0 staining). This is in contrast to our previous studies of Lanzkron et.al in which all PKH26 positive cells were collected after two days post transplant. Thus in Lanzkron et.al. the % positive cells at 48 hours post transplant was 0.43% whereas in our current method the % positive cells represents 0.3% or less of the total.

Viability was determined by propidium iodide. We estimate that our limiting dilution resulted in the injection of 0.5-0.6 viable cells/animal. A group of thirty lethally irradiated recipients received such a transplant along with 2×10⁴ unstained female R/O cells in order to provide short-term but not long-term reconstitution (Jones et al., 1996). In additional experiments, to be absolutely certain that the mice received one male donor derived, passaged, PKH26+ cell, rather than using limiting dilution, a single cell visualized under the microscope was drawn up and delivered directly to a syringe. This cell was then injected along with 2×10⁴ female rotor off cells in a total volume of 500 ul.

Engraftment and FISH Analysis

At five and 11 months post transplant, surviving mice underwent retro-orbital bleeds to assess the percent of donor cell engraftment in the blood. At 11 months recipients were sacrificed. Colony assays were performed with bone marrow by routine methods. Colonies from primary long term survivors or peripheral blood samples from primary and secondary (Table 1) surviving female hosts were collected and FISH for the Y chromosome was done as previously described (Jones et al., 1996; Hawkins et al., 1992). Also, at 11 months 1×10⁶ marrow cells from each host were transplanted into additional groups of four female hosts (for a total of 4×10⁶ cells). FISH analysis was performed two and four months post transplant for the presence of male cells.

After formalin fixation and paraffin embedding, tissues of the five 11 month engrafted mice were analyzed for the Y chromosome. The identification of epithelial cell specific proteins while performing FISH is difficult due to the extensive protease digestion required for FISH, which obliterates antigenic sites needed for antibody binding. Therefore, we used a two step procedure to identify specific cell types and to determine which are male-derived cells as described previously (Theise, et.al., 2000b). First, immunoperoxidase staining using Cam5.2, an antibody against shared epitopes of cytokeratins 8, 18, and 19, or AE1/3, a monoclonal antibody cocktail specific for high molecular weight cytokeratins, was used to label epithelial cells, specifically. After counter-staining with hematoxylin, the sections were color photographed at 20× magnification, and printed as 5×7-inch hard copy pictures obtained. The second step of analysis involved FISH staining for Y-chromosome. Double immunohistochemical staining for cytokeratins and CD11b, a macrophage-specific antigen, was accomplished by adding an incubation step for the biotinylated anti-CD11b rat monoclonal antibody followed by colorization with Fuchsin Red chromogen (DAKO Inc).

For double FISH for Y-chromosome and surfactant-B (SPB) mRNA, slides containing 3 micron tissue sections were deparaffinized and digested with 100 ug/ml proteinase K with 0.05% SDS at 45° C. Genomic DNA probes were prepared based on mRNA sequence for mouse surfactant protein B (SPB, accession: S78114). Primer pairs were synthesized at positions 3758-3781/4064-4041, 8020-8043/8500-8478, 3141-3164/3801-3778, 7849-7871/8500-8478 in the SPB sequenc and PCR products were labeled by incorporation of digoxigenin-dUTP. Mouse Y probe was labeled by PCR using biotin-dUTP. PCR products were then partially digested with DNase I. For each slide, 20 ng dig-labeled surfactant probe and 10 ng biotin-labeled Y chromosome probe were precipitated together with mouse Cot1 DNA (GibcoBRL, Life Technologies, Frederick Md.), resuspended in 10 uL hybridization buffer (50% formamide) and denatured. Slides were denatured 8 minutes at 86° C., and hybridized overnight at 37° C. Posthybridization washes were done at 37° C., followed by antibody detection, using 10 ug/ml protein solutions in 4×SSC. The first detection step included antidigoxigenin and equal amounts of avidin-FITC mixed with avidin Cy5; the second step included sheep antimouse Cy3. The FITC signal enabled visualization of the Y-chromosome signals whereas the Cy5 signal (infrared) was used to provide better signal to noise ratios during image capturing (tissue autofluorescence is higher through the green filter than the Cy5 filter). After washing, slides were mounted in DAPI antifade.

Tissue Analysis and Cell Counts

Counting of Y-positive nuclei was accomplished by systematically examining the FISH stained tissue, field by field, under 60× magnification, using an Olympus Provis (Tokyo, Japan) microscope equipped with a cooled CCD camera (Quantix Corp., Cambridge, Mass.) and specialized software (PSI Inc, League City, Tex.). Autofluorescence was excited at 488 nm, and emission was collected above 515 nm. The rhodamine signal was excited at 568 nm and emission collected above 585 nm. Images were pseudocolored using image processing software (Adobe Photoshop, San Jose Calif.). Cell counts were obtained by first counting all of the Y-chromosome positive cells in a defined area on the tissue, and then counting the total number of cells in that area using the 5×7 immunostained photographs. To compensate for undercounting of Y-positive nuclei due to partial nuclear sampling in tissue sections, cell counts were normalized to the percentage of Y-positive cells seen in the normal male tissue.

### REFERENCES

Abonour, R., Williams, D. A., Einhorn, L., Hall, K. M., Chen, J., Coffman, J., Traycoff, C. M., Bank, A., Kato, I., Ward, M., Williams, S. D., Hromas, R., Robertson, M. J., Smith, F. O., Woo, D., Mills, B., Srour, E. F., and Cornetta, K. (2000). Efficient retrovirus-mediated transfer of the multidrug resistance 1 gene into autologous human long-term repopulating hematopoietic stem cells. Nat Med 6, 652-8.

Alison, M. R., Poulsom, R., Jeffery, R., Dhillon, A. P., Quaglia, A., Jacob, J., Novelli, M., Prentice, G., Williamson, J., and Wright, N. A. (2000). Hepatocytes from non-hepatic adult stem cells. Nature 406, 257.

Bhatia, M., Bonnet, D., Murdoch, B., Gan, O. I., and Dick, J. E. (1998). A newly discovered class of human hematopoietic cells with SCID-repopulating activity. Nat. Med. 4, 1038-1045.

Bjornson, C. R., Rietze, R. L., Reynolds, B. A., Magli, M. C., and Vescovi, A. L. (1999). Turning brain into blood: a hematopoietic fate adopted by adult neural stem cells in vivo. Science 283, 534-7.

Craddock, C. F., Nakamoto, B., Andrews, R. G., Priestley, G. V., and Papayannopoulou, T. (1997). Antibodies to VLA4 integrin mobilize long-term repopulating cells and augment cytokine-induced mobilization in primates and mice. Blood 90, 4779-4788.

Donnelly, D. S., Zelterman, D., Sharkis, S., and Krause, D. S. (1999). Functional activity of murine CD34+ and CD34−hematopoietic stem cell populations. Exp Hematol 27, 788-96.

Eglitis, M. A., and Mezey, E. (1997). Hematopoietic cells differentiate into both microglia and macroglia in the brains of adult mice. Proc Natl Acad Sci 94, 4080-5.

Ferrari, G., Cusella-DeAngelis, G., Coletta, M., Paolucci, E., Stornaiuolo, A., Cossu, G., and Mavilio, F. (1998). Muscle regeneration by bone marrow-derived myogenic progenitors. Science 279, 1528-530.

Goodell, M. A., Brose, K., Paradis, G., Conner, A. S., and Mulligan, R. C. (1996). Isolation and functional properties of murine hematopoietic stem cells that are replicating in vivo. J. Exp. Med. 183, 1797-1806.

Harrison, D. E., Stone, M., and Astle, C. M. (1990). Effects of transplantation on the primitive immunohematopoietic stem cell. J Exp Med 172, 431-437.

Hawkins, A. L., Jones, R. J., Zehnbauer, B. A., Zicha, M. S., Collector, M. I., Sharkis, S. J., and Griffin, C. A. (1992). Fluorescence in situ hybridization to determine engraftment status alter murine bone marrow transplant. Cancer Genet Cytogenet 64, 145-148.

Healy, L., May, G., Gale, K., Grosveld, F., Greaves, M., and Enver, T. (1995). The stem cell antigen CD34 functions as a regulator of hemopoietic cell adhesion. Proc. Natl. Acad. Sci. USA 92, 12240-44

Ito, M., and Kedes, L. (1997). Two-step delivery of retroviruses to postmitotic terminally different cells. Hum. Gene Ther. 8, 57-63.

Jackson, K. A., Mi, T., and Goodell, M. A. (1999). Hematopoietic potential of stem cells isolated from murine skeletal muscle. Proc Natl Acad Sci 96, 14482-6.

Jones, R. J., Celano, P., Sharkis, S. J., and Sensenbrenner, L. L. (1989). Two phases of engraftment established by serial bone marrow transplantation in mice. Blood 73, 397-401.

Jones, R. J., Collector, M. I., Barber, J. P., Vala, M. S., Fackler, M. J., May, W. S., Griffin, C. A., Hawkins, A. L., Zehnbauer, B. A., Hilton, J., Colvin, O. M., and Sharkis, S. J. (1996). Characterization of mouse lymphohematopoietic stem cells lacking spleen colony-forming activity. Blood 88, 487-91.

Krause, D. S., Ito, T., Fackler, M. J., Collector, M. I., Sharkis, S. J., and May, W. S. (1994). Characterization of murine CD34, a marker for hematopoietic progenitor and stem cells. Blood 84, 691-701.

Lagasse, E., Connors, H., Al-Dhalimy, M., Reitsma, M., Dohse, M., Osborne, L., Wang, X., Finegold, M., Weissman, I. L., and Grompe, M. (2000). Purified hematopoietic stem cells can differentiate into hepatocytes in vivo. Nat Med 6, 1229-1234.

Lanzkron, S. M., Collector, M. I., and Sharkis, S. J. (1999). Hematopoietic stem cell tracking in vivo: a comparison of short-term and long-term repopulating cells. Blood 93, 1916-21.

Magdaleno, S. M., Barrish, J., Finegold, M. J., and DeMayo, F. J. (1998). Investigating stem cells in the lung. Adv Pediatr 45, 363-96.

Moll, R., Franke, W. W., Schiller, D. L., Geiger, B., Krepler, R. (1982). The catalog of human cytokeratins: Patterns of expression in normal epithelia, tumors and cultured cells. Cell 31, 11-24.

Morel, F., Szilvassy, S., Travis, M., Chen, B., Galy, A. (1996). Primitive hematopoietic cells in murine bone marrow express the CD34 antigen. Blood, 88, 3774-3784.

Nakamura, Y., Ando, K., Chargui, J., Kawada, H., Sato, T., Tsuji, T., Hotta, T., and Kato, S. (1999). Ex vivo generation of CD34 (+) cells from CD34 (−) hematopoietic cells. Blood 94, 4053-4059.

Nolta, J. A., Crooks, G. M., Overell, W., Williams, D. E., and Kohn, D. B. (1992). Retroviral vector-mediated gene transfer into primitive human hematopoietic progenitor cells: effects of mast cell growth factor (MGF) combined with other cytokines. Exp. Hematol. 20, 1065-71.

Nolta, J. A., Dao, M. A., Wells, S., Smogorzewska, E. M., and Kohn, D. B. (1996). Transduction of pluripotent human hematopoietic stem cells demonstrated by clonal analysis after engraftment in immune-deficient mice. Proc. Natl. Acad. Sci. 93, 2414-19.

Oosterwijk, J. C., Mesker, W. E., Ouwerkerk-van Veizen, M. C., Knepfle, C. F., Wiesmeijer, K. C., van den Burg, M. J., Beverstock, G. C., Bernini, L. F., van Ommen, G. J., Kanhai, H. H., and Tanke, H. J. (1998). Development of a preparation and staining method for fetal erythroblasts in maternal blood: simultaneous immunocytochemical staining and FISH analysis. Cytometry 32, 170-7.

Osawa, M., Hanada, K., Hamada, H., and Nakauchi, H. (1996). Long-term lymphohematopoietic reconstitution by a single CD34-low/negative hematopoietic stem cell. Science 273, 242-245.

Pazouki, S., Hume, R., and Burchell, A. (1996). A rapid combined immunocytochemical and fluorescence in situ hybridization method for the identification of human fetal nucleated red blood cells. Acta Histochem 98, 29-37.

Petersen, B. E., Bowen, W. C., Patrene, K. D., Mars, W. M., Sullivan, A. K., Murase, N., Boggs, S. S., Greenberger, J. S., and Goff, J. P. (1999). Bone marrow as a potential source of hepatic oval cells. Science 284, 1168-70.

Rosen, J. M., Zahnow, C., Kazansky, A., and Raught, B. (1998). Composite response elements mediate hormonal and developmental regulation of milk protein gene expression. Biochem Soc Symp 63, 101-13.

Sato, T., Laver, J. H., and Ogawa, M. (1999) Reversible Expression of CD34 by Murine Hematopoietic Stem Cells. Blood 94, 2548-2554.

Shivdasani, R. A., and Orkin, S. H. (1996). The transcriptional control of hematopoiesis. Blood 87,4025-39.

Sieweke, M. H., and Graf, T. (1998). A transcription factor party during blood cell differentiation. Curr Opin Genet Dev 8, 545-51.

Spangrude, G. J., Brooks, D. M., and Tumas, D. B. (1995). Long-term repopulation of irradiated mice with limiting numbers of purified hematopoietic stem cells: In vivo expansion of stem cell phenotype but not function. Blood 85, 1006-1016.

Sun, T-T., Eichner, R., Schermer, A., Cooper, D., Nelson W. G., Weiss, R. A. (1984). Classification, expression, and possible mechanisms of evolution of mammalian epithelial keratins: A unifying model. Canc Cells 1, 169-76.

Suzuki, A., Andrew, D. P., Gonzalo, J.-A., Fukumoto, M., Spellberg, J., Hashiyama, M., Takimoto, H., Gerwin, N., Webb, I., Molineux, G., Amakawa, R., Tada, Y., Wakeham, A., Brown, J., McNiece, I., Ley, K., Butcher, E.C., Suda, T., Gutierrez-Ramos, J. C., and Mak, T. W. (1996). CD34-deficient mice have reduced eosinophil accumulation after allergen exposure and show a novel crossreactive 90-kD protein. Blood 87, 3550-3562.

Taylor, G., Lehrer, M. S., Jensen, P. J., Sun, T. T., and Lavker, R. M. (2000). Involvement of follicular stem cells in forming not only the follicle but also the epidermis. Cell 102, 451-61.

Theise, N. D., Badve, S., Saxena, R., Henegariu, O., Sell, S., Crawford, J. M., and Krause, D. S. (2000a). Derivation of hepatocytes from bone marrow cells in mice after radiation-induced myeloablation. Hepatology 31, 235-40.

Theise, N. D., Nimmakayalu, M., Gardner, R., Illei, P. B., Morgan, G., Teperman, L., Henegariu, O., and Krause, D. S. (2000b). Liver from bone marrow in humans. Hepatology 32, 11-6.

Travis, E. L., Peters, L. J., McNeill, J., Thames, H. D., Jr., and Karolis, C. (1985). Effect of dose-rate on total body irradiation: lethality and pathologic findings. Radiother Oncol 4, 341-51.

Zahnow, C. A., Younes, P., Laucirica, R., and Rosen, J. M. (1997) Overexpression of C/EBPbeta-LIP, a naturally occurring dominant-negative transcription factor, in human breast cancer. J Natl Cancer Inst 89, 1887-91.

Zanjani, E. D., Almeida-Porada, G., Livingston, A. G., and Ogawa, M. (1998). Human bone marrow CD34− cells engraft in vivo and undergo multilineage expression that includes giving rise to CD34+ cells. Exp Hematol 26, 353-360.

1. A homogeneous preparation of one or more mammalian hematopoietic stem cells.

2. The preparation of claim 1 which does not cause Graft vs. Host Disease upon transplantation.

3. The preparation of claim 1 wherein the hematopoietic stem cells are 6-8 μm in diameter as measured by forward light scattering.

4. The preparation of claim 1 which comprises a single hematopoietic stem cell.

5. The preparation of claim 4 wherein the single cell is capable of reconstituting all bone marrow derived cells upon serial bone marrow transplantation.

6. The preparation of claim 1 which is self-renewing.

7. A method for isolating a homogeneous preparation of hematopoietic stem cells, comprising the steps of:
fractionating bone marrow cells of a donor mammal via elutriation and collecting cells at a flow rate of 20-35 ml/min to form a fraction of bone marrow cells;
depleting the fraction of cells of lineages selected from the group consisting of: T lymphocytes, B lymphocytes, macrophages, granulocytes, erythroid cells, late progenitor cells and combinations thereof, using antibodies specific for markers of said lineages;
labeling the lineage-depleted fraction with a dye which binds to fatty acids in cell membranes;
injecting intravenously the labeled lineage-depleted fraction into a lethally irradiated first mammalian recipient and permitting the injected cells to home to recipient organs for 2 days;
recovering from the first recipient's marrow via flow cytometry a fraction of dye-containing cells which are as dye-bright as dye-labeled cells before said step of injecting and selecting cells which are 6-8 μm in diameter by forward light scattering, to form a homogeneous preparation of hematopoietic stem cells

8. The method of claim 7 further comprising the step of diluting the recovered cells so that single viable cells are present in separate samples.

9. The method of claim 8 further comprising the step of confirming the presence of single viable cells in diluted samples by direct observation.

10. The method of claim 7 wherein the elutriation is counterflow centrifugal elutriation at 1260×g.

11. The method of claim 7 wherein the dye is PKH26.

12. The method of claim 7 which does not employ a step of selecting cells for expression of aldehyde dehydryogenase.

13. The method of claim 7 wherein the flow rate for collecting cells is 23-28 ml/min.

14. The method of claim 7 wherein the flow rate for collecting cells is 25 ml/min.

15. The method of claim 7 further comprising the step of transplanting the selected cells which are 6-8 μm in diameter into a second mammalian recipient.

16. The method of claim 15 wherein the first mammalian recipient and the second mammalian recipient are the same individual.

17. The method of claim 15 wherein the mammalian donor and the first mammalian recipient are the same individual.

18. The method of claim 15 wherein the mammalian donor and the second mammalian recipient are the same individual.

19. The method of claim 15 wherein the first and second mammalian recipients are the same individual.

20. A method for isolating a homogeneous preparation of hematopoietic stem cells, comprising the steps of:
fractionating bone marrow cells of a donor mammal via elutriation and collecting cells at a flow rate of 20-35 ml/min to form a fraction of bone marrow cells;
depleting the fraction of cells of lineages selected from the group consisting of: T lymphocytes, B lymphocytes, macrophages, granulocytes, erythroid cells, late progenitor cells and combinations thereof, using antibodies specific for markers of said lineages;
labeling the lineage-depleted fraction with a dye which binds to fatty acids in cell membranes;
culturing the labeled, lineage-depleted fraction on an irradiated stromal cell culture for 2 days;
recovering from the cultured, labeled, lineage-depleted fraction via flow cytometry a fraction of dye-containing cells which are as dye-bright as dye-labeled cells before said step of culturing, and selecting cells which are 6-8 μm in diameter by forward light scattering to form a homogeneous preparation of hematopoietic stem cells.

21. The method of claim 20 further comprising the step of diluting the recovered cells so that single viable cells are present in separate samples.

22. The method of claim 21 further comprising the step of confirming the presence of single viable cells in diluted samples by direct observation.

23. The method of claim 20 wherein the elutriation is counterflow centrifugal elutriation at 1260×g.

24. The method of claim 20 wherein the dye is PKH26.

25. The method of claim 20 which does not employ a step of selecting cells for expression of aldehyde dehydryogenase.

26. The method of claim 20 wherein the flow rate for collecting cells is 23-28 ml/mm.

27. The method of claim 20 wherein the flow rate for collecting cells is 25 ml/mm.

28. The method of claim 20 further comprising the step of transplanting the selected cells which are 6-8 μm in diameter into a mammalian recipient.

29. The method of claim 28 wherein the mammalian donor and the mammalian recipient are the same individual.

30. The method of claim 28 wherein the mammalian donor and the mammalian recipient are different individuals.

31. A preparation of cells made by the method of any of claims 7-19.

32. A preparation of cells made by the method of any of claims 20-30.
